(19) United States
(12) Patent Application Publication
(10) Pub. No.: US 2010/0008945 A1
(43) Pub. Date: Jan. 14, 2010

(54) Recombinant Newcastle Disease Virus Expressing H5 Hemagglutinin of Avian Influenza Virus

(76) Inventors: Römer-Oberdörfer Angela, Greifswald-Insel Riems (DE); Jutta Veits, Greifswald-Insel Riems (DE)

Correspondence Address: Intervet/Schering-Plough Animal Health, Patent Dept. K-6-1, 1990, 2000 Galloping Hill Road, Kenilworth, NJ, 07033-0530, US

(21) Appl. No.: 12/293,035

(22) PCT Filed: Mar. 15, 2007
(86) PCT No.: PCT/EP07/52429

§ 371 Date: Sep. 15, 2008

Related U.S. Application Data
(60) Provisional application No. 60/783,193, filed on Mar. 15, 2006.

(30) Foreign Application Priority Data
Mar. 15, 2006 (EP) 06111222.3

(51) Int. Cl. A61K 39/145; A61K 39/12; C12N 15/66; C12N 15/86

(52) U.S. Cl. 424/199.1; 435/320.1

(57) ABSTRACT

The present invention provides a method to produce a recombinant Mononegavirales virus vector harbouring an additional transcription unit comprising a foreign gene operatively linked with an upstream Mononegavirales virus gene start (GS) sequence and a downstream Mononegavirales virus gene end (GE) sequence, characterized in that the foreign gene sequence encodes a protein, which protein contains a stretch of at least three basic amino acids and the nucleotide sequence of the codons encoding these amino acids does not contain a sequence that can be recognized by the viral polymerase of the Mononegavirales virus as a gene end (GE) sequence.

This invention relates to a recombinant Mononegavirales virus vector harbouring an additional transcription unit comprising a foreign gene operatively linked with an upstream Mononegavirales virus gene start (GS) sequence and a downstream Mononegavirales virus gene end (GE) sequence. This invention further relates to a method for the production of such a recombinant Mononegavirales virus vector, and to a vaccine comprising such a recombinant Mononegavirales virus vector

Live viruses that are able to replicate in an infected host induce a strong and long-lasting immune response against their expressed antigens. They are effective in eliciting both humoral- and cell-mediated immune responses, as well as stimulating cytokine and chemokine pathways. Therefore, live, attenuated viruses offer distinct advantages over vaccine compositions based on either inactivated or subunit immunogens which typically largely only stimulate the humoral arm of the immune system.

Over the last decade recombinant DNA technology has revolutionized the field of genetic engineering of the genomes of both DNA and RNA viruses. In particular, it is now possible to introduce foreign genes into the genome of a virus such that upon replication of the new vector virus in a host animal a foreign protein is expressed that can exert biological effects in the host animal. As such, recombinant vector viruses have been exploited not only for the control and prevention of microbial infections, but also for devising target therapies for non-microbial diseases such as malignancies and in gene therapy.

The generation of non-segmented, negative stranded RNA viruses (viruses of the order Mononegavirales) entirely from cloned cDNA by a technique designated as “reverse genetics”, first reported in 1994 (Schnell et al., EMBO J 13, 4195-4203, 1994), has made it possible to use also viruses of the order Mononegavirales (MV) as vectors. Since then, studies have been published that describe the use of many viruses of the order MV as viral vectors to express foreign antigens derived from a pathogen aiming at developing vaccines against that pathogen.

The order of Mononegavirales is classified into four main families: Paramyxoviridae, Rhabdoviridae, Filoviridae and Bomaviridae. Viruses belonging to these families have genomes that are represented by a single, negative (−) sense RNA molecule, i.e. the polarity of the RNA genome is opposite to the polarity of messenger RNA (mRNA) that is designated as plus (+) sense. The classification of the main human and veterinary MV viruses is presented in the table below:

TABLE 1
Classification of the viruses within the order of Mononegavirales

| Family | Genus | Species |
|---|---|---|
| Rhabdoviridae | Lyssavirus | Rabies virus (RV) |
| | Vesiculovirus | Vesicular stomatitis virus |
| | | (VSV) |
| | Novirhabdovirus | Infectious hematopoietic |
| | | necrosis virus (IHNV) |
| Paramyxoviridae | Respirovirus | Sendai virus (SeV) |
| | | Human parainfluenza |
| | | virus type 1 and type 3 |
| | | (hPIV 1/3) |
| | | Bovine parainfluenza virus |
| | | type 3 (bPIV 3) |
| | Morbillivirus | Measles virus (MV) |
| | | Rinderpest virus |
| | | Canine distemper virus |
| | | (CDV) |
| | Rubulavirus | Simian virus 5 (SV-5) |
| | | Human parainfluenza |
| | | virus type 2 (hPIV 2) |
| | | Mumps virus |
| | Avulavirus | Newcastle disease virus |
| | | (NDV) |
| | Pneumovirus | Human respiratory |
| | | syncytial virus (hRSV) |
| | | Bovine respiratory |
| | | syncytial virus (bRSV) |
| Filoviridae | Ebola-like virus | Ebolavirus |
| | | Marburg virus |

The genomic organization and details of the life cycle of viruses of the order MV is well understood these days and is reviewed by various authors (Neumann et al., J. Gen. Virology 83, 2635-2662, 2002; Whelan et al., Curr. Top. Microbiol. Immunol. 203, 63-119, 2004; Conzelmann, K., Curr. Top. Microbiol. Immunol. 203, 1-41, 2004). Although Mononegavirales viruses have different hosts and distinct morphological and biological properties, they have many features in common, such as genomic organization and the elements essential for their typical mode of replication and gene expression, illustrating that they have originated from a common ancestor. They are enveloped viruses that replicate in the cytoplasma of the cell and produce mRNAs that are not spliced.

A Mononegavirales virus consists of two major functional units, a ribonucleoprotein (RNP) complex and an envelope. The complete genome sequences for representative viruses of the genera of all the families mentioned above have been determined. The genomes range in size from about 9.000 nucleotides to about 19.000 and they contain from 5 to 10 genes. The structure and the organization of the genomes of the MV viruses are very similar and are governed by their particular mode of gene expression. All of the MV virus genomes comprise three core genes encoding: a nucleoprotein (N or NP), a phosphoprotein (P) and a RNA-dependent RNA polymerase (L). The viral envelope is composed of a matrix (M) protein and one or more transmembrane glycoproteins (e.g. G, HN and F proteins) that play a role in virus assembly/budding as well as in the cell attachment and/or entry of the virus. Depending on the genus the protein repertoire is extended by accessory proteins that display certain specific regulatory functions in transcription and virus replication or that are involved in virus host reactions (e.g. C, V and NS proteins). The gene order of MV viruses is highly conserved with the core genes N and P, at or near the 3′ terminus and with the large (L) gene at the 5′ distal position. The M, the surface glycoprotein genes, as well as the other accessory genes, are located between the N, P and L genes.

In the RNP complex, the genomic or antigenomic RNA is tightly encapsidated with the N protein and is associated with the RNA-dependent RNA polymerase that consists of the L and P protein. After infection of a cell, the RNP complex, but not the naked RNA genome, serves as a template for two distinct RNA synthesis functions, i.e. transcription of subgenomic mRNAs and replication of full length genomic RNA.

All of the tandemly arranged genes are separated by so called “gene junction” structures. A gene junction comprises a conserved “gene end” (GE) sequence, a non-transcribed “intergenic region” (IGR) and a conserved “gene start” (GS) sequence. These sequences are both sufficient and necessary for gene transcription. During transcription each gene is sequentially transcribed into mRNA by the viral RNA-dependent RNA polymerase that starts the transcription process at the 3′ end of the genomic RNA at the first GS sequence. At each gene junction transcription is interrupted as a result of the disengagement of the RNA polymerase at the GE sequence. Re-initiation of transcription occurs at the subsequent GS sequence, although with a reduced efficiency. As a result of this interrupted process, also designated as a “stop-start” process, attenuation of transcription occurs at each gene junction as a result of which the 3′ proximal genes on a MV virus genome are transcribed more abundantly than successive down stream genes. The modular form of transcription of MV virus genes in which each gene is part of a separate cistron or transcription unit makes these viruses extremely suited for the insertion and expression of foreign genes. Each transcription unit in a MV virus genome comprises the following elements: 3′-GS-open reading frame (ORF)-GE-5′.

At the 3′- and 5′-genomic termini all of the MV virus genomes have a short non-transcribed region called “leader” (about 40-50 nt) and “trailer” (about 20-600 nt), respectively. The leader and trailer sequences are essential sequences that control the replication of genomic RNA, viral encapsidation and -packaging.

The reverse genetics technology and the rescue of infectious MV virus have made it possible to manipulate its RNA genome through its cDNA copy. The minimal replication initiation complex required to synthesize viral RNA is the RNP complex. Infectious MV virus can be rescued by intracellular co-expression of (anti)genomic RNAs and the appropriate support proteins from (T7) RNA polymerase driven plasmids. Since the initial report in 1994 by Schnell et al., 1994 (supra), reliable recovery of many MV virus species has been achieved based on the original protocol (or slight variations thereof).

Newcastle disease and avian influenza are important diseases of poultry, which can cause severe economic losses in the poultry industry worldwide. Newcastle disease virus is a non-segmented, negative stranded RNA virus within the order of MV. The genome, which is about 15 kb in length, contains six genes which encode the nucleoprotein (NP), phosphoprotein and V protein (P/V), matrix (M) protein, fusion (F) protein, hemagglutinin-neuraminidase (HN) protein and RNA-dependent RNA polymerase or large (L) protein. The NDV genes are arranged sequentially in the order 3′-NP-P-M-F-HN-L-5′, and are separated by intergenic regions of different length. All genes are preceded by a gene start (GS) sequence which is followed by a noncoding region, the open reading frame encoding the NDV proteins, a second noncoding region and the gene end (GE) sequence. The length of the NDV genome is a multiple of six which has to be considered for the introduction of foreign genes.

Avian influenza (AI) is a disease of poultry characterized by mild respiratory signs to severe disease with high mortality. The causative agent is an avian influenza A virus (AIV) belonging to the family Orthomyxoviridae. AIV contains eight genomic RNA segments of negative polarity which encode 10 proteins. Based on the antigenicity of the surface glycoproteins hemagglutinin (HA) and neuraminidase (N), AI viruses were subtyped. Up to now, 16 hemagglutinin (H1-H16) and nine neuraminidase (N1-N9) subtypes are known. Antibodies to H and N are important in humoral immune response and inhibit infection or prevent disease.

Avian influenza and Newcastle disease viruses can be grouped into two distinct pathotypes according to their virulence. Symptoms caused by low pathogenic AIV (LPAI) or lentogenic NDV are considered of less relevance. In contrast, highly pathogenic avian influenza (HPAI) and Newcastle disease caused by high virulent viruses (NDV: mesogenic and velogenic strains) are notifiable diseases.

Whereas routine vaccination against NDV with lentogenic NDV strains is performed to protect chicken against highly virulent NDV strains, vaccination against HPAI is not performed in most countries, since HPAI is controlled by an eradication strategy. However, vaccination may be used as a strategy to minimize losses and to reduce the incidence of disease. Immunity induced by vaccines is subtype specific, which means that a subtype H5 vaccine can protect against H5 AIV but not against the other H subtypes. Normally, influenza virus replication is restricted to the lungs because hemagglutinin of LPAI viruses can be cleaved only by tryptase Clara, a serine protease restricted to the lungs. So far, all HPAI viruses have been of H5 and H7 subtype. These HPAI viruses contain multiple basic amino acids at the H cleavage site so that it can be cleaved by ubiquitous furin and subtilisin-like enzymes into the subunits HA1 and HA2. Such viruses can therefore grow in other organs.

Subtype H5 and H7 vaccines can provide protection of chickens and turkeys against clinical signs and death following infection with HPAI. In addition to conventional inactivated oil-based whole AIV, vector virus, subunit protein and DNA vaccines have been shown experimentally to be effective for immunization against AI. Since the advent of reverse genetics for different viruses the generation of recombinant viruses for use as vaccine vectors is an important application. Different recombinant negative-strand RNA viruses expressing foreign proteins have been constructed. Also, the hemagglutinin of AIV was inserted into different vector viruses like the infectious laryngotracheitis virus (ILTV) (Luschow et al., Vaccine 19, 4249-59, 2001), Rinderpest virus (Walsh et al., J. Virol. 74, 10165-75, 2000) and vesicular stomatitis virus (VSV) (Roberts et al., J. Virol. 247, 4704-11, 1998). Also NDV was used for the expression of AIV hemagglutinin. The hemagglutinin gene of influenza A/WSN/33 was inserted between P and M genes of NDV strain Hitchner B1. This recombinant protected mice against lethal infection although there was a detectable weight loss in mice which recovered fully within 10 days Nakaya et al. (J. Virol. 75, 11868-73, 2001). A further recombinant NDV with the same insertion site for the foreign gene expressed the H7 of a LPAI but only 40% of the vaccinated chicken were protected from both velogenic NDV and HPAI (Swayne et al., Avian Dis. 47, 1047-50, 2003).

Influenza HA proteins, are synthesized as precursor proteins (HA₀). The precursopr polypeptide HA₀ is post translationally cleaved at a conserved arginine (Arg) residue into two subunits, Ha₁ and HA₂. This cleavage contributes to the infectivity of the virus. (The more efficient the HA precursor is cleaved the more virulent the virus seems to be). The HA₁-HA₂ junction regions of various influenza viruses have been analyzed. It was discovered that pathogenic strains contain a stretch of basic amino acids adjacent to the cleavage site. (Senne et al., Avian Diseases, 40: 425-437, 1996; Steinhauser, Virology, 258, 1-20, 1999; Kawaoka and Webster, Proc.Natl.Acad.Sci.USA, 85, 1988.).

Especially the highly pathogenic viruses are important in terms of the protection that should be provided by an influenza vaccine.

However, when these types of proteins, that is, proteins like the HA proteins of a pathogenic H5 influenza, that have a basic stretch of amino acids in their sequence, were inserted into a mononegavirales vector, certain problems were encountered.

First the gene sequence seemed instable to a certain extend. After several egg passages of an NDV vector in which an H5 gene had been inserted (NDVH5), spontaneous mutations occurred within the part of the gene sequence encoding the basic stretch of amino acids.

In addition, the coding sequence for the stretch of basic amino acids is likely to contain a sequence that is recognized by a mononegavirus as a gene end (GE) sequence. An overview of GE sequences for mononegavirales is given in Whelan, (Curr. Top. Microbiol. Immunol., 283,61-119, 2004). The GE sequences for monegavirales are characterized by a common conserved sequence: nUUUU.

It was found that parts of the coding sequence for a stretch of basic amino acids, such as present adjacent to the cleavage site of the HA protein of pathogenic H5 type avian influenza virus, resemble the GE sequence recognized by the polymerases of the mononegavirales vector as a GE sequence. This may lead to reduced levels of protein expression of the full length protein, which may affect the efficacy of a vaccine based on such a vector.

It is an object of this invention to provide a recombinant MV virus vector that displays a higher expression level of a protein encoded by a foreign gene inserted into the genome of the vector virus and/or that shows an increased immunogenicity than existing MV virus vectors.

The present inventors have found that this object can be met by a recombinant Mononegavirales virus according to the invention.

The present invention provides a method to produce a recombinant Mononegavirales virus vector harboring an additional transcription unit comprising a foreign gene operatively linked with an upstream Mononegavirales virus gene start (GS) sequence and a downstream Mononegavirales virus gene end (GE) sequence, characterized in that the foreign gene sequence encodes a protein, which protein contains a stretch of at least three basic amino acids, said stretch consisting of Arginine (Arg) and/or Lysine (Lys) residues and containing at least one Lysine, wherein the nucleotide sequence of the foreign gene is selected in such a way that it does not contain a sequence that can be recognized by the viral polymerase of the mononegavirales virus as a gene end (GE) sequence.

A sequence that can be recognized by the viral polymerase of the mononegavirales virus as a gene end sequence is a sequence that would encompass the minimal conserved sequence shared by most GE sequences of mononegavirales, namely a/uCUUUU (in the negative sense, which is the genomic sense for a mononegavirus). In a positive sense (cDNA level) this motive is t/aGAAAA).

For the specific mononegavirales vector used the specific GE sequence known in the art would apply Whelan et al. supra). For NDV for example, the GE sequence listed in Whelan is aaucUUUUUUu. (−sense, which is the genomic sense for mononegavirales). In the +sense (cDNA level), the sequence would thus be characterized by a stretch of adenine residues: gAAAAAA.

In a vector according to the invention such a GE sequence does not occur within the coding sequence of the foreign protein.

There are several options open to the skilled person to achieve this goal. A foreign sequence can be selected that, by nature does not contain sequence that can be recognized by the viral polymerase of the mononegavirales virus as a gene end (GE) sequence.

For example, when the foreign gene sequence encodes a viral protein, the foreign gene may be obtained from a strain of the virus which, by nature, carries a gene encoding this protein of which the coding sequence does not encompass a potential GE sequence (whereas the same gene in another strain of the virus does)

However, since chances are low that such a variant gene sequence can be obtained form a wild type virus, it is preferred to mutate a wild type sequence of the foreign gene by site directed mutagenesis to alter the sequence to such an extend that the potential GE sequence is no longer present.

The resulting recombinant Mononegavirales virus vector wherein the foreign gene encodes a protein which protein contains a stretch of at least three basic amino acids, said stretch consisting of Arginine (Arg) and/or Lysine (Lys) residues and containing at least one Lysine, which foreign gene does not contain a sequence that can be recognized by the viral polymerase of the mononegavirales virus as a gene end sequence, is likewise part of the invention.

The part of the foreign gene that needs to be modified, especially in the case where the foreign protein is the HA protein of a pathogenic H5 avian influenza virus, may be the part of a wild type foreign gene sequence encoding a stretch of at least three basic amino acids.

A stretch of basic amino acids is defined as encompassing at least three amino acids selected from the group consisting of Lysine (one letter code: K) and Arginine (one letter code: R), wherein at least one the amino acid residues is Lysine. This would include the sequence KKK, and combinations of 2K with 1R (KKR, KRK, RKK), and combinations of 1K and 2R (KRR, RKR, and RRK), but also longer stretches of basic amino acids including the specific 3 amino acids sequences mentioned here. As can be seen from Steinhauser et al. (supra) the stretches of basic amino acids adjacent to the cleavage sites in the HA proteins of pathogenic influenza viruses may be longer, and may include sequences like RRKKR, or RRRKK.

Codons encoding Lysine are aaa and aag, whereas codons encoding arginine include aga and agg. It has been found that, especially in the coding sequence for the HA protein of H5 pathogenic avian influenza viruses, the coding sequence encoding the basic stretch of amino acids adjacent to the cleavage site of the protein may contain the GE motive t/aGAAAA.

To modify the GE sequence present within the wild type foreign gene, at least one mutation may be introduced whereby an “A” of the adenine stretch within a GE sequence, having the common motive (t/aGAAAA), originally present in the wild type foreign gene sequence, is replaced by another nucleotide.

In practice, if a mutation is introduced in the “aaaa” tract that is part of a sequence recognized as a GE sequence, at least one of the adenine nucleotides would have to be replaced by another nucleotide (for example a guanidine (“g”)).

When altering the sequence by site directed mutagenesis these alterations preferably are silent (meaning that the mutation(s) do not lead to a mutation at the amino acid level). However mutations at the nucleic acid level that lead to conserved mutations at the amino acid level (meaning that one basic amino acid is replaced by another basic amino acid, e.g. L becomes R, or R becomes L) may also be acceptable. For example, where, in the part of the wild type gene sequence encoding the basic stretch of amino acids, at least one lysine residue is encoded by the codon “aaa” this codon may be mutated into “aag” in the modified foreign gene inserted into a vector of the invention. Or, where in the wild type sequence a particular arginine residue that forms part of the basic stretch of amino acids is encoded by “aga”, this codon may be mutated into “agg” in the gene as it is made part of a vector according to the invention.

At least one “aaa” codon (encoding a lysine) in the wild type sequence may be mutated to create the modified foreign gene sequence that is incorporated into a vector of the invention. Such a mutation may involve replacement of the “aaa” codon by a “aag” codon. Vectors wherein for example the stretch of basic amino acids contains at least two Lysine amino acids in row, and wherein at least one of these lysines is encoded by the codon “aag” are preferred, but two or more mutations may be made. For example, where the coding sequence would contain two “aaa” codons in row, both may be replaced by “aag” codons.

Especially in view of the desired stability of the resulting vector, it is preferred to introduce at least two mutations into the foreign gene. Where the foreign gene contains two “aaa” codons in row, both are preferably mutated, and may be replaced by aag codons instead.

Good results were obtained with vectors wherein the part of the foreign gene encoding the basic stretch of amino acids contained one mutation in each of three or four consecutive codons encoding a basic amino acid. In cases where the basic stretch encompasses only three amino acids, three mutations may be made, but when there are four basic amino acids or more, more mutations may be made.

For example, where the wild type sequence contains the amino acid sequence RRKK, encoded by the nucleotide sequence aga aga aaa aaa (+sense sequence, wherein the potential GE sequence motiv is underlined), this coding sequence may be mutated into agg agg aag aag still encoding RRKK.

The Mononegavirales virus vector preferably is a Newcastle Disease virus (NDV) vector and the foreign gene the HA protein of a pathogenic H5 avian influenza virus.

Good results were obtained with an NDV vector in which a modifed HA gene of a pathogenic H5 avian influenza had been incorporated (NDVH5m).

The HA gene had been incorporated in the intergenic region between the NDV fusion (F) and hemagluttinin-neuraminidase (HN) genes of the NDV vector. The HA gene was a modified H5 gene, wherein the wild type “aga aga aaa aaa” sequence had been changed into a “agg agg aag aag” sequence according to the method of the present invention.

This vector produced significantly more full-length HA transcripts, expressed significantly higher levels of HA and also incorporated more HA proteins in its envelope.

Moreover, NDVH5m stably expressed the modified HA gene for more than 10 egg passages. Immunization of chickens with NDVH5m induced NDV- and AIVH5-specific antibodies and protected chickens against clinical disease after challenge with a lethal dose of velogenic NDV or highly pathogenic AIV, respectively. Remarkably, shedding of influenza virus was not observed. Furthermore, immunization with NDVH5m permitted serological discrimination of vaccinated and AIV field-virus infected animals based on antibodies against the nucleoprotein (NP) of AIV. Therefore, recombinant NDVH5m is suitable as a bivalent vaccine against NDV and AIV, and may be used as marker vaccine for the control of avian influenza (AI).

Methods for the preparation of a recombinant MV virus vector harboring an additional transcription unit comprising a foreign gene are well known in the art.

More in particular, in this method the isolated nucleic acid molecule and the genome of the MV virus are used in their cDNA form (+sense). This allows easy manipulation and insertion of the desired nucleic acid molecules into the viral genome.

In general, various parts of the genome could be used for the insertion of the foreign gene, between two genes, i.e. in intergenic regions (IGR), 3′ or 5′ non-coding regions of a gene as well as 3′ promoter-proximal (before the N/NP genes) or 5′ distal end (after the L genes) of a genome.

The foreign gene could advantageously be inserted before the N/NP gene, between NP-P, P-M, M-G/F, G/F-HN, HN-L and after L gene.

The simplest way is to use an already existing restriction enzyme (RE) recognition sequence at one of these sites by cutting with the enzyme and introducing an appropriate transcription cassette. Since naturally existing restriction enzyme recognition sequences are not always located at the desired location, RE recognition sites could be introduced into the genome conventionally by site directed- or PCR mutagenesis.

The composition of the transcription cassette to be inserted depends on the site of insertion. For example, in case a transcription cassette is inserted into an IGR the cassette may comprise the following elements: 3′ RE recognition site-GS-non coding region-ORF (of foreign gene)-non coding region-GE-RE recognition site 5′.

Alternatively, in case a transcription cassette is introduced into a 5′ non-coding region of a natural MV virus gene the cassette may be composed of: 3′ RE recognition site-GE-IGR-GS-non coding region-ORF (of foreign gene)-non coding region-RE recognition site 5′.

Similarly, in case a transcription cassette is to be introduced into a 3′ non-coding region of a natural MV virus gene the cassette may be composed of 3′ RE recognition site—non coding region-ORF (of foreign gene)-non coding region-GE-IGR-GS-RE recognition site 5′.

The preparation of such transcription cassettes and the insertion thereof into a MV virus genome only involve routine molecular biological techniques, such as exemplified in the literature references listed, and in the present Examples. In particular, techniques such as site-directed- and PCR mutagenesis can be used for this purpose (Peeters et al., 1999, supra; Current Protocols in Molecular Biology, eds.: F. M. Ausubel et al., Wiley N.Y., 1995 edition, pages 8.5.1.-8.5.9; and Kunkel et al., Methods in Enzymology Vol. 154, 376-382, 1987).

More in particular, a recombinant MV virus vector according to the present invention can be prepared by means of the well established “reverse genetics” method that enables the genetic modification of non-segmented, negative stranded RNA viruses of the order MV (reviewed for example by Conzelmann, K. K., Current Topics Microbiol. Immunol. 203, 1-41, 2004; and Walpita et al., FEMS Microbiol. Letters 244, 9-18, 2005).

In this method, an appropriate cell is co-transfected by a vector comprising a cDNA molecule comprising a nucleotide sequence that encodes a full length genome, or, preferably, an antigenome (positive sense) of a MV virus, and one or more vectors comprising the cDNA molecules comprising nucleotide sequences that encode the required support proteins, under conditions sufficient to permit transcription and co-expression of the MV (anti)genome and support proteins and the production of a recombinant MV vector. In this method, the said nucleic acid molecule encoding the full length MV virus (anti)genome comprises an additional transcription unit as defined above.

With vector is meant a replicon, such as a plasmid, phage or cosmid, to which another DNA segment may be attached so as to bring about the replication of the attached DNA segment and its transcription and/or expression in a cell transfected with this vector.

Preferably, the vector for the transcription of the full length genome is a plasmid that comprises a cDNA sequence encoding the (anti)genome of the MV virus, flanked by a T7 polymerase promoter at its 5′ end and a (hepatitis delta) ribozyme sequence at its 3′ end, although a T3 or SP6 RNA polymerase promoter can also be used.

For the intra-cellular expression of the appropriate support proteins use is made, preferably, of plasmids comprising the cDNA sequence encoding these proteins, under the control of appropriate expression control sequences, e.g. a T7 polymerase promoter.

In a particularly preferred method for the preparation of a recombinant MV virus vector according to the present invention, expression plasmids are used that encode the N (or NP), P and L proteins of the MV virus.

The amounts or ratios of transfected support plasmids to be used in this reverse genetic technology cover a broad range. Ratios for the support plasmids N:P:L may range from about 20:10:1 to 1:1:2 and efficient transfection protocols for each virus are known in the art.

An exact copy of the genomic RNA is made in a transfected cell by the combined action of the T7 RNA polymerase promoter and the ribozyme sequence, and this RNA is subsequently packaged and replicated by the viral support proteins supplied by the co-transfected expression plasmids.

It is preferred that a T7 polymerase enzyme is provided by a recombinant vaccinia virus that infects the transfected cell, in particular by the vaccinia virus vTF7-3, yet also other recombinant pox vectors, such as fowl pox virus, e.g. fpEFLT7pol, or other viral vectors may be used for the expression of T7 RNA polymerase.

Separation of the rescued virus from vaccinia virus can easily be accomplished by simple physical techniques, such as filtration. For rescue of Sendai virus or NDV, rescue can be achieved by inoculation of the supernatant of transfected cells in embryonated eggs.

In an even more preferred embodiment cell lines are used for the transfection of the transcription- and expression vectors that constitutively express the (T7) RNA polymerase and/or one or more of the required support proteins.

For example, rescue of Measles virus can be achieved in a human embryo kidney cell line, 293-3-46, that expresses both T7 RNA polymerase and Measles virus support proteins N and P (Radecke et al., EMBO J. 14, 5773-5784, 1995). Another very useful cell line that can be used advantageously in the present invention is based on BSR cells expressing the T7 RNA polymerae, i.e. cell line BSR-T7/5 (Buchholz et al., J. Virol. 73, 251-259, 1999).

Furthermore, more detailed information concerning the reverse genetics technology to be used herein for the preparation of a MV virus according to the present invention is disclosed in the review by Conzelmann, K. K. (supra) and Example 1.

The ability of recombinant MV virus vectors to stably express foreign genes has resulted in the development of vectors for both prophylactic and therapeutic applications.

In a recombinant MV virus vector according to the present invention the foreign gene can vary depending on the specific MV virus vector species and the application of the vector virus.

The foreign gene may encode an antigen of an (other) microbial pathogen (e.g. a virus, bacterium of parasite), especially the foreign gene encodes an antigen of a pathogen that is able to elicit a protective immune response.

For example, heterologous gene sequences that can be inserted into the virus vectors of the invention include, but are not limited to influenza virus glycoprotein genes, in particular, H5 and H7 hemagglutinin genes of avian influenza virus, genes derived from Infectious Bursal Disease Virus (IBDV), specifically VP2 of (IBDV), genes derived from Infectious Bronchitis Virus (IBV), feline leukemia virus, canine distemper virus, equine infectious anemia virus, rabies virus, ehrlichia organism, in particular Ehrlichia canis, respiratory syncytial viruses, parainfluenza viruses, human metapneumoviruses and measles virus.

Alternatively, the foreign gene may encode a polypeptide immune-modulator that is able to enhance or modulate the immune response to the virus infection, for example by co-expressing a cytokine such as an interleukin (e.g. IL-2, IL-12, IFN-γ, TNF-α or GM-CSF).

The order of MV includes both viruses that are able to replicate in humans and animals, or in both (e.g. rabies virus and Newcastle disease virus). Therefore, the foreign gene can be selected from a wide variety of human and veterinary microbial pathogens.

Although all MV viruses can be used as a vector virus in the present invention, in a preferred embodiment of the invention the recombinant MV virus vector is a virus of the family Rhabdoviridae, preferably of the genus Lyssavirus or Novirhabdovirus, more preferably of the species rabies virus or IHNV, respectively.

In an also preferred embodiment the recombinant MV virus is a virus of the family Paramyxoviridae, preferably of the genuses Respovirus, in particular the species hPIV3 or bPIV3; Morbillivirus, in particular the species CDV; Pneumovirus, in particular the species RSV; and Avulavirus, in particular the species NDV.

In a particularly preferred embodiment of the present invention a recombinant MV virus vector is provided wherein the virus is Newcastle disease virus (NDV). As NDV is able to replicate in both humans and animals, in particular poultry, more in particular, chickens, a recombinant NDV vector according to the invention may comprise a foreign gene that encodes an antigen of a pathogen, in particular of a respiratoty pathogen, or an immune-modulator that is capable of eliciting an appropriate immune response in humans or any of these animals.

Reverse genetics methods for the genetic manipulation of NDV have been disclosed specifically for NDV by Peeters et al. (J. Virology 73, 5001-5009, 1999), Römer-Oberdörfer et al. (J. Gen. Virol. 80, 2987-2995, 1999), and in the review by Conzelmann, K. K. (supra). Furthermore, it is also known that NDV can be used as a vector for the expression of foreign genes, for example, for the eliciting of an immune response in animals infected with the NDV vector (Nakaya et al., 2001, supra) and Swayne et al., Avian Dis. 47, 1047-50, 2003).

A foreign gene can advantageously be introduced into a NDV genome at various positions as outlined in general for MV viruses above. In particular, in a recombinant NDV vector according to the invention, a foreign gene (as part of an appropriate transcription unit) can be inserted between the following NDV genes: NP-P, P-M, M-F, F-HN, HN-L and at the 3′ proximal- and 5′ distal locus (Zhao et al., 2003, supra; Nakaya et al., 2001, supra), preferably in the 3′ proximal, P-M, M-F and F-HN regions, the F-HN region being most preferred.

In a particular embodiment of the invention a recombinant NDV vector is provided wherein the additional transcription unit is located between the F-HN genes.

A recombinant NDV vector according to the present invention can advantageously be used to induce an immune response in poultry, in particular chickens, against other pathogens. Therefore, the recombinant NDV vector, preferably comprises a foreign gene that encodes a protective antigen of an avian pathogen, in particular of influenza virus, marek's disease virus (MDV), infectious laryngotracheitis virus (ILTV), infectious bronchitis virus (IBV), infectious bursal disease virus (IBDV), chicken anemia virus (CAV), reo virus, avian retro virus, fowl adeno virus, turkey rhinotracheitis virus (TRTV), E. coli, Eimeria species, Cryptosporidia, Mycoplasms such as M. gallinarum, M. synoviae and M. meleagridis, Salmonella-, Campylobacter-, Omithobacterium (ORT) or Pasteurella sp.

More preferably, the recombinant NDV vector comprises a foreign gene that encodes an antigen of AIV, MDV, ILTV, IBV, TRTV, E. coli, ORT or Mycoplasma.

In particular, the recombinant NDV vector mutant comprises a hemagglutinin (HA) gene of an influenza virus, preferably of an avian influenza virus (AIV), more preferably of a highly pathogenic AIV, in particular of H5 or H7 AIV.

In principle, the HA gene of all (avian) influenza strains can be used in this invention. The nucleotide sequences of many HA gene have been disclosed in the art and the relevant HA genes can be retrieved from nucleic acid sequence databases, such as GenBank or the NCBI database.

The hemagglutinin (HA) gene of the recently isolated, highly pathogenic H5N2 subtype AIV A/chicken/Italy/8/98 can advantageously be used as a foreign gene in the present invention as outlined above. The gene is reverse transcribed, cloned in the eukaryotic expression vector pcDNA3 (Invitrogen), and sequenced (Lüschow et al., Vaccine, vol. 19, p. 4249-4259, 2001, and GenBank Accession No. AJ305306). From the obtained expression plasmid pCD-HA5 the HA gene can be obtained by amplification by using specific primers that generate artificial RE recognition sites that allows insertion of the HA gene in NDV genomic sequences.

In a further embodiment the HA gene of the highly pathogenic H7N1 subtype AIV A/chicken/Italy/445/99 can be used as a foreign gene in the present invention as outlined above. The HA gene is reverse transcribed, and amplified by PCR. The 1711 bp product is cloned in the SmaI-digested vector pUC18 (Amersham) and sequenced (Veits et al., J. Gen. Virol. 84, 3343-3352, 2003; and GenBank Accession No. AJ580353).

In a particularly advantageous recombinant MV virus vector according to the present invention the MV vector virus is attenuated, that is to say, the vector virus is not pathogenic for the target animal or exhibits a substantial reduction of virulence compared to the wild-type virus. Many MV viruses used herein as virus vectors have a long safety record as live attenuated vaccines such as the measles virus and NDV, whereas other viruses, such as SeV and VSV are considered non-pathogenic to humans. In addition, conventional techniques exist to obtain and screen for attenuated viruses that show a limited replication or infectivity potential. Such techniques include serial (cold) passaging the virus in a heterologous substrate and chemical mutagenesis.

A recombinant NDV vector according to the invention can be derived from any conventional ND vaccine strain. Examples of such suitable NDV strains present in commercially available ND vaccines are: Clone-30®, La Sota, Hitchner B1, NDW, C2 and AV4; Clone-30® being the preferred strain.

It has also been found by the present inventors that a recombinant MV virus vector according to the present invention is able to induce a protective immune response in animals.

Therefore, in another embodiment of this invention a vaccine against a microbial pathogen is provided that comprises a recombinant MV virus vector as defined above in a live or inactivated form, and a pharmaceutically acceptable carrier or diluent.

A vaccine according to the invention can be prepared by conventional methods such as those commonly used for the commercially available live- and inactivated MV virus vaccines.

Briefly, a susceptible substrate is inoculated with the recombinant MV virus vector and propagated until the virus replicated to a desired titre after which the virus containing material is harvested. Subsequently, the harvested material is formulated into a pharmaceutical preparation with immunizing properties.

Every substrate which is able to support the replication of the recombinant MV virus vector can be used in the present invention. As a substrate host cells can be used from both prokaryotic- and eukaryotic origin, depending on the MV virus. Appropriate host cells may be vertebrate, e.g. primate cells. Suitable examples are; the human cell lines HEK, WI-38, MRC-5 or H-239, the simian cell line Vero, the rodent cell line CHO, BHK, the canine cell line MDCK or avian CEF or CEK cells.

A particularly suitable substrate on which a recombinant NDV vector according to the present invention can be propagated are SPF embryonated eggs. Embryonated eggs can be inoculated with, for example 0.2 ml NDV containing allantoic fluid comprising at least 10^{2.0} EID₅₀ per egg. Preferably, 9- to 11-day old embryonated eggs are inoculated with about 10^{5.0} EID₅₀ and subsequently incubated at 37° C. for 24 days. After 2-4 days the ND virus product can be harvested preferably by collecting the allantoic fluid. The fluid can be centrifuged thereafter for 10 min. at 2500 g followed by filtering the supernatant through a filter (100 μm).

The vaccine according to the invention comprises the recombinant MV virus vector together with a pharmaceutically acceptable carrier or diluent customary used for such compositions.

The vaccine containing the live virus can be prepared and marketed in the form of a suspension or in a lyophilized form. Carriers include stabilizers, preservatives, and buffers. Diluents include water, aqueous buffer and polyols.

In another aspect of the present invention a vaccine is provided comprising the recombinant MV virus vector in an inactivated form. The major advantages of an inactivated vaccine are its safety and the high levels of protective antibodies of long duration that can be induced.

The aim of inactivation of the viruses harvested after the propagation step is to eliminate reproduction of the viruses. In general, this can be achieved by well known chemical or physical means.

If desired, the vaccine according to the invention may contain an adjuvant. Examples of suitable compounds and compositions with adjuvant activity for this purpose are aluminum hydroxide, -phosphate or -oxide, oil-in-water or water-in-oil emulsion based on, for example a mineral oil, such as Bayol F® or Marcol 52® or a vegetable oil such as vitamin E acetate, and saponins.

The administration of a vaccine according to the invention may be by any of the well known effective forms and may depend on the type of MV virus vector. Suitable modes of administration include, parenteral-, intranasal, oral and spray vaccination.

A NDV vector vaccine according to the invention is preferably administered by the inexpensive mass application techniques commonly used for NDV vaccination. For NDV vaccination these techniques include drinking water and spray vaccination.

A vaccine according to the invention comprises an effective dosage of the recombinant MV virus vector as the active component, i.e. an amount of immunizing MV virus material that will induce immunity in the vaccinated birds against challenge by a virulent microbial organism. Immunity is defined herein as the induction of a significant higher level of protection in a population of humans or animals against mortality and clinical symptoms after vaccination compared to an unvaccinated group. In particular, the vaccine according to the invention prevents a large proportion of vaccinated humans or animals against the occurrence of clinical symptoms of the disease and mortality.

Typically, the live vaccine can be administered in a dose of 10^{2.0}-10^{8.0} tissue culture/embryo infectious dose (TC/EID₅₀), preferably in a dose ranging from 10^{4.0}-10^{7.0} TC/EID₅₀. Inactivated vaccines may contain the antigenic equivalent of 10^{4.0}-10^{9.0} TC/EID₅₀.

The invention also includes combination vaccines comprising, in addition to the recombinant MV virus vector according to the invention, a vaccine strain capable of inducing protection against a further pathogen.

### BRIEF DESCRIPTION OF THE FIGURES

FIG. 1

Construction of recombinant NDV expressing AIV H5. Transcription control signals are marked by a grey triangle for transcription start and a grey rectangle for transcription stop sequences. The HN ORF of NDV was substituted by the H5 ORF of AIV and the HN gene was inserted subsequently as described in material and methods (steps A-C). Recombinant NDVH5 (step D) contains the authentic HA sequence, whereas recombinant NDVH5m carries an HA in which the cleavage site sequence was altered by silent mutation (steps E-F).

FIG. 2

Northern blot analyses of transcripts produced by NDV recombinants. Total RNA of CEK cells infected with NDV Clone 30 (NDV), NDVH5, NDVH5m or AIV A/chicken/Italy/8/98 (H5N2) and non infected cells (NI) were prepared after 8 hours of infection. RNA was separated in denaturing agarose gels, transferred to nylon membranes, and hybridized with ³²P-labeled gene-specific antisense cRNAs of NDV-F (left), AIV-H5 (middle) and NDV-HN (right). Six μg of the respective RNA was subjected to the Northern hybridization, except for the AIV lane in the middle blot where only 2 μg was loaded to avoid excessive AIV signal. Sizes of the RNA marker are indicated in kilo bases (kb) on the left.

FIG. 3

Western blot analyses of proteins produced in infected cells or incorporated into NDV recombinants expressing the HA of AIV. Lysates of infected cells (A) or purified virions (B) of NDV Clone 30, NDVH5, NDVH5m or AIV (H5N2) were either incubated with an AIV subtype H5-specific antiserum (α-AIV; upper panels) or with a NDV-specific antiserum (α-NDV; lower panels). Binding was visualized by chemiluminescence after incubation with peroxidase-conjugated secondary antibodies. Locations of marker proteins are indicated on the left and the uncleaved (HA₀) and processed forms (HA₁, HA₂) of AIV hemagglutinin are indicated on the right. In AIV infected cells or virions additional viral proteins corresponding to NP/NA and M are also detectable.

FIG. 4

HA specific antibodies measured by HI test (A, grey bars) or by IF test (A, white bars). Mortality rates and clinical indices of chickens immunized once (B, C) or two times (D) with NDVH5m (H5m) and control animals (C) after challenge with the velogenic NDV Herts (B) or highly pathogenic H5N2 AIV (C, D). The animals were observed daily for a period of 10 days for clinical signs and classified as healthy (0), ill (1), severely ill (2), or dead (3). A clinical index was calculated which represents the mean value of all chickens per group for this period.

FIG. 5

Serological examinations by NP-ELISA. Sera of chickens were investigated by an indirect NP-ELISA at a dilution of 1:500. The raised P/N-ratio were plotted for sera of chickens immunized with rNDV/AIVH5-BMUT collected on day 21 post immunization (p.i.) and 21 d after subsequent AIV challenge (p.c.). The cut-off value of 2.0 is marked by a dotted line.

### EXAMPLES

### Example 1

### Construction of NDV Vectors Comprising Non-Modified (Wild Type) H5 Gene and Modified H5 Gene and Expression of the Protein

Newcastle disease virus (NDV) expressing avian influenza virus (AIV) hemagglutinin (HA) of subtype H5 was constructed by reverse genetics. A cloned full-length copy of the genome of the lentogenic NDV strain Clone 30 was used for insertion of the open reading frame encoding the HA of the highly pathogenic (HP) AIV isolate A/chicken/Italy/8/98 (H5N2, Genbank accession number AJ305306) in the intergenic region between the NDV fusion (F) and hemagglutinin-neuraminidase (HN) genes.

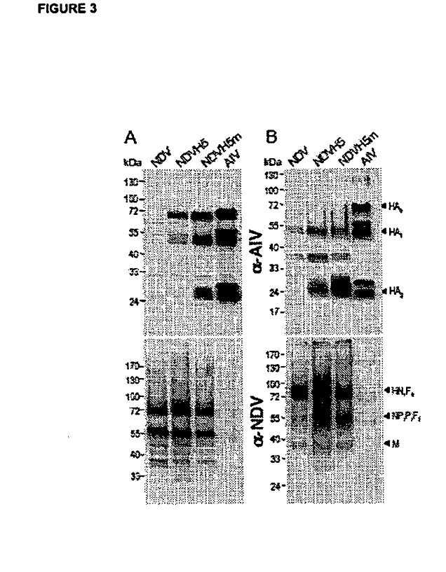

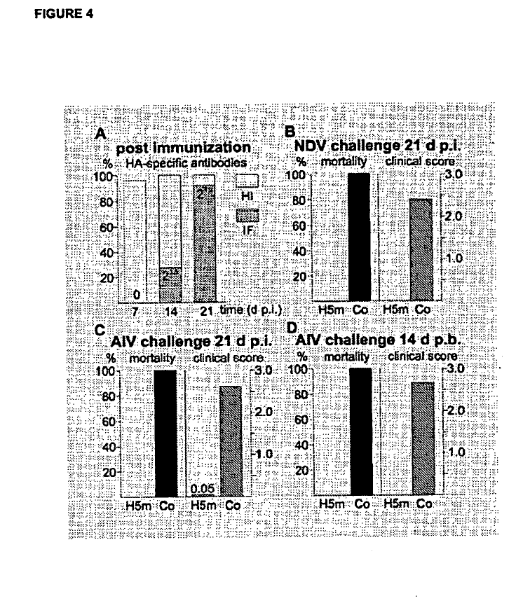

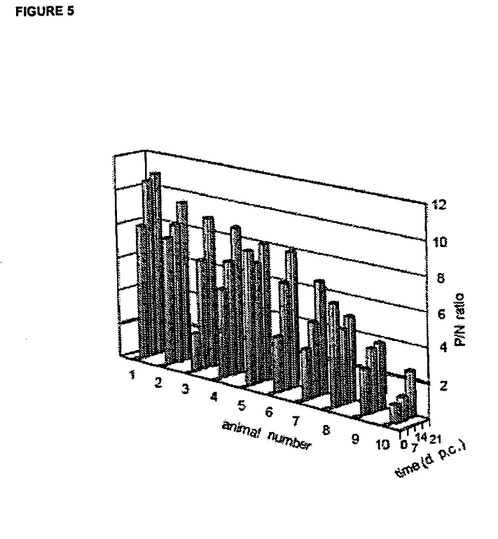

After three egg passages, the titer of NDVH5 recombinant was similar to that of the parental virus Clone 30 (10⁹ TCID₅₀/ml), and the presence of the inserted H5 gene was confirmed by RT-PCR. To verify correct transcription of the foreign gene, Northern blot analysis was performed (FIG. 2). Although the H5 ORF in the recombinant NDV and AIV (H5N2) is identical, the H5 ORF cloned into NDV is flanked by approximately 270 nucleotides derived from the non-coding sequences of HN gene. Therefore, the size difference between the NDVH5 and AIV mRNAs is in good agreement with the expected size of ˜2 kb and 1.7 kb mRNAs, respectively (FIG. 2, middle). However a second transcript of about 1 kb in NDVH5 appeared. (FIG. 2, middle, lane 3).

As revealed by Northern blot analysis, recombinant NDVH5m produced only the expected size of 2 kb H5 transcript (FIG. 2, middle, lane 4), confirming that the short transcript in NDVH5 terminates at the HA cleavage site and most likely represents only the HA, region.

As a result of the modification, NDVH5m produced 1.8-fold more full-length HA (HA₀) than NDVH5. Full-length HA transcripts in AIV infected cells were 3.4- and 6-fold more than that of NDVH5m and NDVH5, respectively. Unlike the H5 cleavage site sequence of NDVH5, which has been altered within 5 passages, the NDVH5 sequence in this region remained stable throughout the tested 10 passages (Table 1). Hybridization with NDV F and HN probes confirmed correct transcription of the flanking NDV genes, as there was no size difference to F and HN mRNA of the parent NDV Clone 30 (FIG. 2). Furthermore, only a slight reduction in the transcription rate of the HN gene was observed as a result of the insertion of the HA ORF (FIG. 2, right).

To confirm the specific expression of AIV H5 by the NDV recombinants, infected CEF cells were subjected to indirect IF test. Incubation with a NDV-specific antiserum revealed a pronounced fluorescence in cells infected with NDV Clone 30, NDVH5 and NDVH5m but not in cells infected with AIV-H5N2. An AIV subtype H5-specific antiserum on the other hand showed specific expression of H5 in cells infected with AIV, NDVH5 and NDVH5m, whereas cells infected with the parent NDV were negative. Although the intensity of H5 expression by AIV infected cells is considerably higher than that of both recombinants, the level of H5 expression by the NDV recombinants was found to be remarkable.

In agreement with this, only 3-fold more NDVH or NDVH5 RNA amounts were loaded onto the RNA-gels to obtain comparable hybridization signals, shown in FIG. 2 (middle), indicating that AIV produced approximately 3-fold more transcripts and presumably so much more protein. Interestingly, H5 expression by NDVH5m infected cells appears more efficient than in cells infected with NDVH5, which could be attributed to the silent mutation abrogating premature transcription termination of H5.

### Example 2

### The AIV H5 Protein is Incorporated into the Envelope of NDV Particles

Previous studies showed that incorporation of foreign proteins into the envelope of unrelated viruses could occur passively in the absence of specific incorporation signals (Kretzschmar, E. et al., 1997, J. of Virology, vol. 71, p. 5982-5989). Since the efficiency of such passive incorporation mainly depends on the expression level of the proteins, we determined whether the HA protein expressed by the recombinant viruses was properly cleaved and also incorporated into the envelope of NDV recombinants (FIG. 3). In cells infected by both recombinants, the AIV subtype H5-specific antiserum detected three proteins of approximately 70, 50 and 25 kDa presumably representing the uncleaved HA₀ and the cleaved HA₁ and HA₂ proteins (FIG. 3A). This demonstrates that the HA expressed by the recombinants is accessible to proteolytic enzymes. The total HA protein produced in cells infected with NDVH5 virus was remarkably less than that of NDVH5m and the HA2 protein in NDVH5 infected cells is barely visible. As expected, no reactivity could be detected in NDV Clone 30 infected cells, whereas all corresponding HA protein species were detected in AIV-H5N2 infected cells. As the AI specific serum was raised against the whole virus, other protein species presumably corresponding to NP/NA and M were also detected in AIV infected cells (FIG. 3). Interestingly, Western blot analyses of purified virions indicated that the HA protein was Incorporated efficiently into the envelope of both recombinants (FIG. 3B). Although, more NDVH5 viral protein was subjected to Western blot analysis, the amount of HA2 protein incorporated into NDVH5 was still considerably lower than the HA2 in NDVH5m virions. This demonstrates that NDVH5 produces and incorporates much less HA2 protein, presumably due to the availability of less protein as a result of the premature termination at the cleavage site.

### Example 3

### Recombinant NDV Carrying AIV HA Protein is Safe in Chickens

One of the sensitive ways of measuring the degree of virulence of a given NDV isolate is by assessing the pathogenicity of the virus for 1-day-old chickens after intracerebral inoculation (CEC (1992) Official Journal of the European Community L 260, 1-20.). The most virulent viruses will give indices that approach the maximum score of 2.0, whereas lentogenic strains will give values close to 0.0. Since the hemagglutinin of AIV is an important virulence determinant, the intracerebral pathogenicity indices (ICPI) for NDVH5 and NDVH5m were determined to evaluate if expression of H5 of a HPAI virus alters NDV virulence. The ICPI values were 0.0, out of a maximum possible score of 2 for both recombinants, demonstrating that expression of the AIV H5 in addition to its own proteins did not noticeably affect NDV virulence. For comparison, the ICPI of a lentogenic NDV vaccine strain has to be below 0.5 for a possible live virus vaccine use.

### Example 4

### Recombinant NDV Expressing the AIV HA Protein Protects Chickens Against NDV and AIV Challenges

Because of a higher expression of H5 protein by NDVH5m, only this recombinant was tested in the animal experiments. Recombinant NDVH5 was thus administered to 25 three-week-old chickens at a dose of 10⁶ EID₅₀ per animal by oculonasal route. During the observation period, all the animals remained healthy without any adverse reactions or any clinical signs of disease. As determined by the HI test, AIV H5-specific antibodies were first detectable on day 14 in 28% of the sera and increased to 92% on day 21 after vaccination (FIG. 4A). An earlier onset of immunity against AI was, however, detected in 96% of sera at 7 days post-immunisation using an indirect IF test (FIG. 4A).

To determine the protective effect of a single vaccination, groups of 5 and 10 vaccinated animals, together with appropriate number of control animals, were subjected to a first round of lethal challenges against NDV and AIV, respectively. Not unexpectedly, 100% of the vaccinated animals were protected against lethal velogenic NDV challenge, whereas all control animals died within 4 days exhibiting typical signs of ND (FIG. 4B). The highly pathogenic AIV-H5N2 challenge infection caused severe disease in non-immunized chickens with a mortality rate of 100 %. In contrast, all animals of the NDVH5m immunized group survived the lethal dose of highly pathogenic AIV. Seven out of ten chickens remained completely healthy, whereas three animals exhibited very mild respiratory symptoms. However, the resulting clinical score of 0.05 was negligible compared to the clinical score of 2.61 for the control group (FIG. 4C). To determine the effect of a booster vaccination in protecting chickens against AI, the remaining ten chickens received a second immunization on day 42 after the first vaccination and challenged two weeks thereafter. All animals were completely protected against clinical disease after a lethal dose of the homologous HPAI virus, whereas all control animals developed severe disease and died within 4 days resulting in a clinical score of 2.66 (FIG. 4D).

### Example 5

### NDV-AI Vaccine Reduces AIV Shedding

For an AI vaccine to be successful, the vaccine should be effective in preventing virus shedding besides being safe and efficacious in preventing disease. To determine the amount of virus shedding, tracheal and cloacal swabs were subjected to quantitative real time RT-PCR at different times post challenge. Viral RNA was detected in all non-immunized but challenged chickens of both control groups on day 2 post challenge. The threshold cycle (Ct) values of swabs of the control chickens of the first and second AIV challenge ranged between 32.2.-38.1 and 29.2-35.5, respectively. Since all control animals died within 4 days of challenge, no further test could be performed on this group. In contrast, no vRNA was detected in most of the immunized animals (49 out of 60 swabs).

### Example 6

### NP-ELISA Detects Circulating AIV

One of the greatest fears of routine vaccination of poultry is the probability that vaccination could enable the virus to circulate undetected among birds. Since the recombinant NDV vaccine only contains the HA gene, an ELISA based on the highly immunogenic nucleoprotein gene was employed to analyze the sera collected from vaccinated animals before and at different times after challenge. Whereas antibodies against AIV NP were absent in sera of all animals before challenge infection, NP seroconversion could be detected in 90% and 100% of the chickens on day 7 and 21 after AIV challenge, respectively (FIG. 5). This demonstrates that the NP-based ELISA test enables not only differentiation of vaccinated animals from infected ones, but also facilitates detection of any circulating virus among vaccinated birds.

### Example 7

### Material and Methods for Examples 1-6

Viruses and Cells:

The recombinant NDV based on the vaccine strain of Clone-30 has been described previously (Römer-Oberdörfer, A., Mundt, E., Mebatsion, T., Buchholz, U. J. & Mettenleiter, T. C. (1999), J. Gen. Virol. 80 (Pt 11), 2987-2995.). The influenza virus isolate A/chicken/Italy/8/98 (H5N2) was kindly provided by I. Capua. The velogenic NDV strain Herts 33/56 and the NDV Clone 30 vaccine (Nobilis®) were obtained from Intervet Int. B V, Boxmeer, The Netherlands. The viruses were propagated in specific pathogen free (SPF) 10-day-old embryonated chicken eggs. BSR-T7/5 cells stably expressing phage T7 RNA polymerase (Buchholz, U. J., Finke, S. & Conzelmann, K. K. (1999) J. Virol. 73, 251-259) were used to recover infectious NDV from cDNA. Primary chicken embryo fibrobasts (CEF), primary chicken embryo kidney (CEK) cells or quail muscle cells (QM9-R) were used to investigate protein expression and virus replication.

Construction of Recombinant Viruses Expressing the AIV H5 Gene

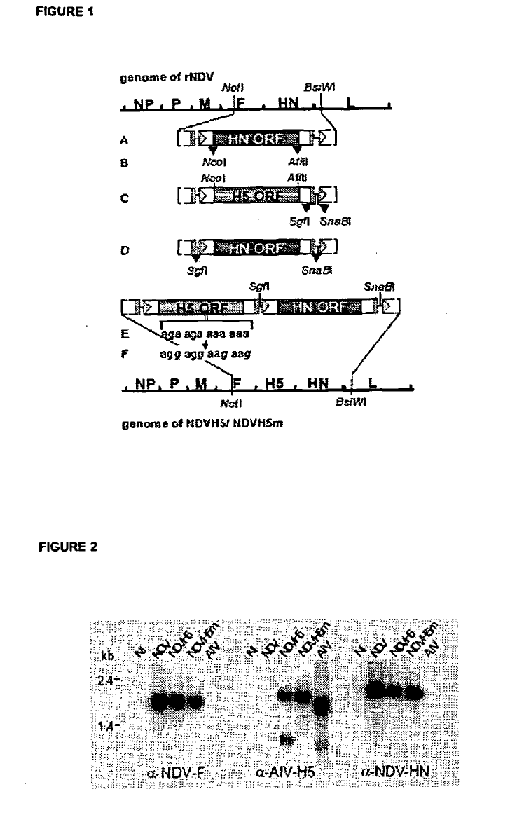

The plasmid pfINDV-1, expressing the full-length antigenomic RNA of Clone 30 (Römer-Oberdörfer, A., Mundt, E., Mebatsion, T., Buchholz, U. J. & Mettenleiter, T. C. (1999) J. Gen. Virol. 80 (Pt 11), 2987-2995) was used to introduce the AIV H5 gene. First, a NotI/BsiWI-fragment (nt 4953-8852) of Clone 30 genome was cloned into pUC18 plasmid (step A, FIG. 1). Newly created NcoI and AfIII sites were then introduced (step B) using primers MP1 (5′-gacaacagtcctcaaccatggaccgcgccg-3′, SEQ ID NO: 1) and MP2 (5′-ctggctagt tgagtcaattcttaaggagttggaaagatggc-3′, SEQ ID NO: 2). The AIV H5 open reading frame (ORF), which has been amplified from plasmid pCD-HA5 (Lüschow, D., Werner, O., Mettenleiter, T. C. & Fuchs, W. (2001) Vaccine 19, 4249-4259) by specific primers containing artificial NcoI or AfIII restriction sites (PH5F2: 5′-ccftccatggagaaaatagtgcttc-3′, SEQ ID NO: 3, and PH5R2: 5′-cctccftaagtataaftgactcaattaaatgcaaattctgcactgcaatgatcc-3′, SEQ ID NO: 4), was used to substitute the HN ORF of Clone 30 after digestion with NcoI and AfIII (step C). In addition, new SgfI- and SnaBI-sites were introduced in the intergenic region in front of the L gene (step C) using primers MP3 (5′-caaaacagctcatggtacgtaatacgggtaggacatgg-3′, SEQ ID NO: 5) and MP4 (5′-gaaaaaactaccggcgatcgctgaccaaaggacgatatacggg-3′, SEQ ID NO: 6). Similar sites flanking the HN gene (step D) were generated using primers MP3 and MP5 (5′-gaaaaaactaccggcgatcgctgaccaaaggacgatatacggg-3′, SEQ ID NO: 7) for the purpose of cloning the HN gene after the H5 ORF (step E). The H5 cleavage sequence resembling transcription termination sequence of NDV was modified by silent mutation using primer MPH5F2 (5′-ggaatgtccctcaaagaaggaggaagaagagaggactatttggggc-3′, SEQ ID NO: 8) as shown in step F of FIG. 1. Finally, the NotI/BsiWI-fragment of pfINDV-1 was substituted by a similar fragment obtained from steps E or F to create full-length clones NDVH or NDVHm, respectively (FIG. 1). The length of the resultant clones (17196 nucleotides) is divisible by six thereby fulfilling the “rule of six”. All mutagenesis reactions described here were done using the Quik Change II XL site directed mutagenesis kit (Stratagene).

Transfection and Virus Recovery:

To recover recombinant NDV expressing the H5 of AI, the full-length clones together with plasmids expressing the NP, P and L proteins were transfected into BSR-T7 cells using Lipofectamine 2000 (Invitrogen) at the DNA:Lipofectamine 2000 rate of 1:1,5. Virus propagation and confirmation of the recover of infectious virus were essentially carried out as described previously (Römer-Oberdörfer, 1999, supra; Engel-Herbert, I., Werner, O., Teifke, J. P., Mebatsion, T., Mettenleiter, T. C. & Romer-Oberdorfer, A. (2003) J. Virol. Methods 108, 19-28).

Northern Blot Analyses:

CEK cells were infected with NDV Clone 30, NDVH5, NDVH5m or AIV-H5N2 at a multiplicity of infection (MOI) of 10 per cell and incubated for 8 h at 37° C. Total RNA of infected and uninfected cells was prepared (Chomczynski, P. & Sacchi, N. (1987) Analytical Biochemistry 162, 156-159), separated in denaturing agarose gels and hybridized with radiolabeled cRNAs as described elsewhere (Fuchs, W. & Mettenleiter, T. C. (1996) J. Gen. Virol. 77 (Pt 9), 2221-2229). Plasmids containing the open reading frames of AIV-H5N2 hemagglutinin, NDV Clone 30 F and HN were used for in vitro transcription of ³²P-labeled cRNA (SP6/T7 Transcription kit, Roche).

Western Blot Analyses:

CEK cells were infected at an MOI of 5 with NDV Clone 30, NDVH5, NDVH5m or AIV-H5N2 and incubated for 30 h at 37° C. Lysates of infected cells or virions purified by continuous sucrose gradient (30-60%) were separated by SDS-PAGE and transferred to nitrocellulose filters (Trans-Blot SD cell, Bio-Rad). Blots were incubated with a polyclonal rabbit antiserum against NDV, or a polyclonal chicken antiserum against AIV of the subtype H5 (Intervet Int. BV, Boxmeer, NL). Binding of peroxidase-conjugated species-specific secondary antibodies (Dianova) was detected by chemiluminescence using SuperSignal West Pico Chemiluminescent Substrate (Pierce) on X-ray films (Hyperfilm MP, Amersham).

Animal Experiments:

The safety of the NDV recombinants was assessed by determining the intracerebral pathogenicity index (ICPI) in 1-day-old chickens according as described in the European Community Council Directive (CEC (1992) Official Journal of the European Community L 260, 1-20.). Vaccination experiments were carried out by administering 10⁶ EID₅₀ of NDVH5m oculonasally into 25 three-week-old SPF chickens. To evaluate the protective ability of the recombinant after a single vaccination, five of the vaccinates together with 5 additional controls were challenged with 10^{5.3} ELD₅₀ of NDV strain Herts 33/56 intramuscularily three weeks post vaccination. Likewise, 10 of the vaccinates together with nine additional control animals were challenged oculonasally with 10^{7,7} EID₅₀ of the highly pathogenic AIV-H5N2. The remaining ten chickens received a second immunization six weeks after the first vaccination and were subjected to a similar AI challenge together with five additional control animals two weeks thereafter. After immunizations and challenge infections, all birds were observed daily for a period of 10 days for clinical signs.

Virus Shedding Analysis by Real-Time RT-PCR:

Oropharyngeal and cloacal swabs were collected to analyze AIV shedding by real-time RT-PCR on days 2, 4, 8 and 14 after challenge. RNA from oropharyngeal or cloacal swabs was prepared either by Tecan-Automat using the Nucleo Spin kit (Macherey-Nagel) or manually using the viral RNA kit (Qiagen). For the detection of AIV shedding after challenge infection, the Influenza A virus real-time RT-PCR method based an amplification of the M gene was used (18). The RNA extraction and inhibition factors during the RT-PCR were checked by a heterologous internal control system (19). The duplex assay was performed on the MX3000p (Stratagene) cycler using the one step RT-PCR kit (Superscript™ III One-Step RT-PCR system with Platinum® Taq DNA Polymerase (Invitrogen)). The temperature profile was 30 min 50° C., 2 min 94° C., followed by 42 cycles of 30 sec at 94° C., 30 sec at 57° C. and 30 sec at 68° C.

HI and NP-ELISA:

To determine the presence of NDV and AIVH5 antibodies, blood samples were collected at 0, 7, 14 and 21 days and subjected to hemagglutination-inhibition (HI) test as described in the European Community Council Directive (CEC (1992) Official Journal of the European Community L 260, 1-20; CEC (1992) Official Journal of the European Communities L 167, 1-1620,21). For assessing the presence of AIV-H5 antibodies after immunization, the sera were additionally used for indirect immunofluorescence (IF) by incubating 1:100 dilution of the sera with AIV infected CEF. Antibodies against AIV nucleoprotein (NP) were investigated by an indirect enzyme-linked immunosorbent assays (ELISAs) based on the nucleoprotein (NP-ELISA). For this purpose, a purified recombinant baculovirus-derived gluthatione-S-transferase-NP fusion protein, encompassing the complete coding region of the AIV NP gene, was used as antigen. Sera diluted 1:300 in PBS containing 0.05% Tween 20 were investigated in duplicate. Binding of secondary POD-conjugated goat-α-chicken IgG (H+L) (ROCKLAND) antibodies was detected by a colour reaction using o-phenylenediamine and Absorbtion was measured at 492 nm.

### Example 8

### Construction of NDV Vectors Comprising a Modified H7 Gene

In experiments essentially similar to those described above, an NDV vector construct was made carrying a modified H7 gene. The insert was derived from the HP H7N1 AIV isolate: A/chicken/Italy/445/99, the sequence of which is published in GenBank under accession number AJ 580353; see also: Veits, J. et al. 2003 (J. of Gen. Virol., vol. 84, p. 3343). The H7 gene was inserted into the NDV vector in between the F and HN genes, and was flanked by non-coding sequences from the HN gene.

A potential gene end sequence is present after the cleavage site region of the H7 HA gene. The original sequence in nucleotides 1195-1206: ata gaa aaa act, encoding the amino acids IEKT, was mutated into: ata gag aag act, stil encoding IEKT.

This resulted in a stable and improved expression and presentation of the modified H7 sequence via the NDV vector, compared to expression of an un-modified H7 gene.

1. A method to produce a recombinant Mononegavirales virus vector harboring an additional transcription unit comprising a foreign gene operatively linked with an upstream Mononegavirales virus gene start (GS) sequence and a downstream Mononegavirales virus gene end (GE) sequence, characterized in that the foreign gene sequence encodes a protein which protein contains a stretch of at least three basic amino acids, said stretch consisting of Arginine (Arg) and/or Lysine (Lys) residues and containing at least one Lysine, wherein the nucleotide sequence of the foreign gene is selected in such a way that it does not contain a sequence that can be recognized by the viral polymerase of the mononegavirales virus as a gene end (GE) sequence.

2. A method according to claim 1, characterized in that the part of a wild type foreign gene sequence encoding said stretch of at least three basic amino acids is modified by site directed mutagenesis to eliminate any sequence that can be recognized by the viral polymerase of the mononegavirales virus as a gene end (GE) sequence, to create a modified foreign gene sequence.

3. A method according to claim 2, characterized in that within a t/aGAAAA sequence originally present in the wild type foreign gene sequence, at least one mutation is introduced whereby an “A” of the adenine stretch is replaced by another nucleotide.

4. A method according to claim 3, characterized in that the mutation(s) are silent.

5. A method according to claim 3, characterized in that at least one aaa codon is replaced by an aag codon.

6. A method according to claim 3, characterized in that at least two point mutations are made.

7. A method according to claim 3, characterized in that at least two aaa codons are replaced by an aag codon.

8. A method according to claim 3, characterized in that the coding sequence aga aga aaa aaa is replaced by the coding sequence agg agg aag aag.

9. A method according to claim 1, characterized in that Mononegavirales virus vector is a Newcastle Disease virus (NDV) vector and the foreign gene encodes the HA protein of a pathogenic H5 avian influenza virus.

10. A recombinant Mononegavirales virus vector harboring an additional transcription unit comprising a foreign gene operatively linked with an upstream Mononegavirales virus gene start (GS) sequence and a downstream Mononegavirales virus gene end (GE) sequence, characterized in that the foreign gene encodes a protein which protein contains a stretch of at least three basic amino acids, said stretch consisting of Arginine (Arg) and/or Lysine (Lys) residues and containing at least one Lysine, which foreign gene does not contain a sequence that can be recognized by the viral polymerase of the mononegavirales virus as a gene end sequence.

11. A recombinant Mononegavirales virus vector according to claim 10, characterized in that the foreign gene encodes the hemagglutinin (HA) protein of a pathogenic avian influenza virus, wherein the stretch of basic amino acids contains at least two Lysine amino acids in row, and wherein at least one of these lysines is encoded by the codon aag.

12. A recombinant Mononegavirales virus vector according to claim 10, characterized in that the stretch of basic amino acids contains the amino acid sequence Arg Lys Lys, encoded by the nucleotide sequence aggaagaag.

13. A vaccine against a microbial pathogen comprising a recombinant MV virus vector according to claim 10 and a pharmaceutically acceptable carrier or diluent.

14. A method according to claim 4, characterized in that at least one aaa codon is replaced by an aag codon.

15. A method according to claim 4, characterized in that at least two point mutations are made.

16. A method according to claim 5, characterized in that at least two point mutations are made.

17. A method according to claim 4, characterized in that at least two aaa codons are replaced by an aag codon.

18. A method according to claim 5, characterized in that at least two aaa codons are replaced by an aag codon.

19. A method according to claim 6, characterized in that at least two aaa codons are replaced by an aag codon.

20. A method according to claim 4, characterized in that the coding sequence aga aga aaa aaa is replaced by the coding sequence agg agg aag aag.


SEQUENCE LISTING (8 sequences)

SEQ ID NO: 1 - DNA, 30 bp, Artificial
  cloning primer
gacaacagtcctcaaccatggaccgcgccg

SEQ ID NO: 2 - DNA, 41 bp, Artificial
  cloning primer
ctggctagttgagtcaattcttaaggagttggaaagatggc

SEQ ID NO: 3 - DNA, 25 bp, Artificial
  cloning primer
ccttccatggagaaaatagtgcttc

SEQ ID NO: 4 - DNA, 54 bp, Artificial
  cloning primer
cctccttaagtataattgactcaattaaatgcaaattctgcactgcaatgatcc

SEQ ID NO: 5 - DNA, 38 bp, Artificial
  cloning primer
caaaacagctcatggtacgtaatacgggtaggacatgg

SEQ ID NO: 6 - DNA, 43 bp, Artificial
  cloning primer
gaaaaaactaccggcgatcgctgaccaaaggacgatatacggg

SEQ ID NO: 7 - DNA, 43 bp, Artificial; identical to SEQ ID NO: 6
  cloning primer

SEQ ID NO: 8 - DNA, 46 bp, Artificial
  cloning primer
ggaatgtccctcaaagaaggaggaagaagagaggactatttggggc